(19) United States
(12) Patent Application Publication
(10) Pub. No.: US 2019/0348163 A1
(43) Pub. Date: Nov. 14, 2019

(54) PERSONNEL TRACK AND TRACE AUDIT SYSTEM FOR HEALTHCARE

(71) Applicants: Allen B. Kantrowitz, Miami, FL (US); In Ki Mun, Nanuet, NY (US)

(72) Inventors: Allen B. Kantrowitz, Miami, FL (US); In Ki Mun, Nanuet, NY (US)

(21) Appl. No.: 16/407,879

(22) Filed: May 9, 2019

Related U.S. Application Data
(60) Provisional application No. 62/668,902, filed on May 9, 2018.

(51) Int. Cl. G16H 15/00; G06K 7/10; G06Q 30/04; G06N 5/02; G06F 9/451

(52) U.S. Cl. CPC G16H 15/00; G06K 7/10297; G06F 9/453; G06N 5/02; G06Q 30/04

(57) ABSTRACT

An automated system for medical billing is provided that includes one or more radio frequency identification (RFID) sensors positioned in various rooms or areas of a medical facility. The RFID sensors are in communication with a setS of individually coded radio frequency identification (RFID) tags assigned to each member of a team of healthcare workers, pieces of equipment, and patients seeking medical care in the medical facility. A software platform run on a processor determines probable tasks performed by the members of the healthcare team based on the locations and time spent by the healthcare members with the one or more pieces of equipment and the one or more patients being attended to, and where the determined probable tasks are associated with a corresponding billing codes. A method of system usage is also provided.

### RELATED APPLICATIONS

This application is a non-provisional application that claims priority benefit of US Provisional Application Ser. No. 62/668,902 filed 9 May 2018; the contents of which are hereby incorporated by reference.

### TECHNICAL FIELD

The present invention generally relates to the field of medical billing, and more specifically to a system and method that improves the timeliness, convenience, and accuracy of capturing information for medical billing.

### BACKGROUND

Medical practice management software (PMS) is a category of healthcare software that deals with the day-to-day operations of a medical practice. PMS software frequently allows users to capture patient demographics, schedule appointments, maintain lists of insurance payors, perform billing tasks, and generate reports.

PMS systems are designed for small to medium-sized medical offices. Versions of PMS software are designed for or used by third-party medical billing companies. PMS is often divided among desktop-only software, client-server software, or Internet-based software. The desktop-only software is intended to be used only on one computer by one or a handful of users sharing access. Client-server software typically necessitates that the practice acquire or lease server equipment and operate the server software on that hardware, while individual users' workstations contain client software that accesses the server. An advantage of client-server software is in allowing multiple users to share the data and the workload, while a major disadvantage is the cost of running the server. Internet-based software that is based in the cloud is a relatively newer PMS based offering. Internet or cloud based software decreases the need for the medical office or practice to run their own server and worry about security and reliability. However, Internet based software removes patient data from the practice's premises, which can present a security risk.

Practice management software is often connected to electronic medical records (EMR) systems. While some information in a PMS and an EMR overlaps—for example, patient and provider data—in general the EMR system is used for the assisting the practice with clinical matters, while PMS is used for administrative and financial matters. Medical practices often hire different vendors to provide the EMR and PMS systems. The integration of the EMR and PMS software is considered one of the most challenging aspects of the medical practice management software implementation.

Medical billing is a process that involves health care providers and insurance companies (payers), and pertains to the payment to the health care providers for medical services rendered to clients of the insurance companies by the health care providers. A billing interaction begins with an office visit or hospital admission of a patient, when a physician or their staff will typically create or update the patient's medical record. After the doctor sees the patient, the diagnosis and procedure codes are assigned. The procedure codes assist the insurance company in determining coverage and medical necessity of the services. Once the procedure and diagnosis codes are determined, a medical biller will transmit the claim to the insurance company. The insurance company will then review the claim and pay based on their findings and percentage payout policies.

Currently, bill coding is typically performed with software packages that generate user interfaces (UI) that utilize long drag down menus of patients and services. The use of multiple and lengthy pull down menus is tedious, time consuming, and prone to error, and practically proper billing event capture is not done routinely with lost billing and inaccurate time logs that miss services performed by medical personnel.

Point of care (POC) functionality often uses a radio frequency network that allows a handheld device used by the caregiver to communicate in real-time with a health care information system, and as a result, there is no need to upload or download information to/from the device. Bedside verification systems interface with a healthcare facilities information system to obtain data regarding patient identification, physician computerized order entry, and medication administration records (MAR) with the bedside unit dose administration event. Patients and unit dose medications are often associated and identified with a set of unique barcodes that may be scanned by the handheld device to confirm a patient's identity and medication information against the available data in the MAR to ensure safe medication administration and for reducing preventable medication errors.

Radio frequency identification (RFID) tags may be used in conjunction with radio frequency networks for tracking and determining the location of people, equipment, and objects in a location or a facility. Radiofrequency identification (RFID) tags are broadly classed as to passive and active tags. While a passive tag lacks a power supply in electrical communication with the tag, an active identification tag has a coupled power supply and actively broadcasts a signal. Generally, a passive tag tends to be less expensive, more compact and has a longer operating lifetime than a comparable active tag, at the expense of requiring more complex tag interrogation systems.

While there are available medical practice management software and point of care systems for managing, recording, billing, and administering patient care, there continues to be a need for improved methods and systems that improve the timeliness, convenience, and accuracy of capturing information of medical billing.

### SUMMARY OF THE INVENTION

An automated system for medical billing is provided that includes one or more radio frequency identification (RFID) sensors positioned in various rooms or areas of a medical facility. The RFID sensors are in communication with: a first set of individually coded radio frequency identification (RFID) tags assigned to each member of a team of healthcare workers in the medical facility, a second set of individually coded RFID tags assigned to one or more pieces of equipment in the medical facility, and a third set of individually coded RFID tags assigned to one or more patients seeking medical care in the medical facility. A software platform run on a processor that collects information from the one or more RFID sensors determines probable tasks performed by the members of the healthcare team based on the locations and time spent by the healthcare members with the one or more pieces of equipment and the one or more patients being attended to, and where the determined probable tasks are associated with a corresponding billing codes. A prospective bill is generated that is quicker to edit and confirm that de novo generation. A method of system usage is also provided.

### BRIEF DESCRIPTION OF THE DRAWINGS

The present invention is further detailed with respect to the following drawings that are intended to show certain aspects of the present invention, but should not be construed as a limit on the practice of the invention, wherein:

FIG. 1 is a block diagram of a medical facility configured with a system of radio frequency identification (RFID) readers to determine the positions and locations of medical personnel, medical equipment, and patients in accordance with embodiments of the invention;

FIG. 2 illustrates a user interface (UI) for entering medical tasks in accordance with embodiments of the invention; and

FIG. 3 is a schematic diagram illustrating an overall view of communication devices, computing devices, and mediums for implementing embodiments of the invention.

### DETAILED DESCRIPTION OF THE INVENTION

The present invention has utility as a system and method that improves the timeliness, convenience, and accuracy of capturing information for medical billing. Embodiments of the inventive tracking and auditing system employ an array of radio frequency ID sensors deployed in a healthcare environment to determine the position of healthcare workers, patients, and equipment each of which carry a unique RFID badge. While such a system can be used to generate an audit trail of proximity and position of each badge, embodiments of the present invention uses the positional data to address medical billing. As noted above, bill coding is currently done on software platforms that utilize long drag down/pull down menus of patients and services that is time consuming to do, and practically it is not done routinely with lost billing and inaccurate time logs that miss services.

Embodiments of the present invention, link a unique RFID badge to that of a caregiver to a central database and provides suggestions of activities or tasks and efforts being performed by the caregiver based on the amount of time and position spent in the proximity of a patient or a piece of medical equipment. For example, a physician spends time visiting a newly admitted patient and their portable computing device illustratively including a tablet or smartphone afterwards asks “If you performed an admission consult, click here to confirm; if not, {back to a drag down list}”. The implication is that if the systems informed suggestion is correct, a repetitive time saving is achieved by eliminating the need to navigate through a series of pull down menus. In a similar manner, if the physician or patient is near a magnetic resonance imaging (MRI) machine for a period of time, the system may make an informed suggestion to the physician or technician as to a billing event related to performance of an MM. Lastly, a physician sitting in front of computer provides RFID proximity and the computer provides the files being reviewed and prompts “are you reviewing the chart of patient X? if yes click here, else {drag down menus are presented}”.

In a specific inventive embodiment artificial intelligence (AI) may be utilized to improve the informed suggestions and become more efficient, and also learn the flow patterns of healthcare workers to offer hospital administration ways to minimize travel time by reconfiguring the layout of the hospital work space. The sensor data and curated billing codes are readily provided to a commercial AI service, for example as provided by SAS or IBM Watson, to refine the system billing code assignments based on repeated input as to the behavior and resulting billing codes of medical staff, equipment usage, and patient activity. Furthermore, AI may be used to remember previous manually entered activities or tasks that were entered via drag/pull down menus or typed in based on the previous location of the user when entered. Artificial intelligence may then be used to make informed and intelligent suggestions to the user for tasks and corresponding billing code suggestions.

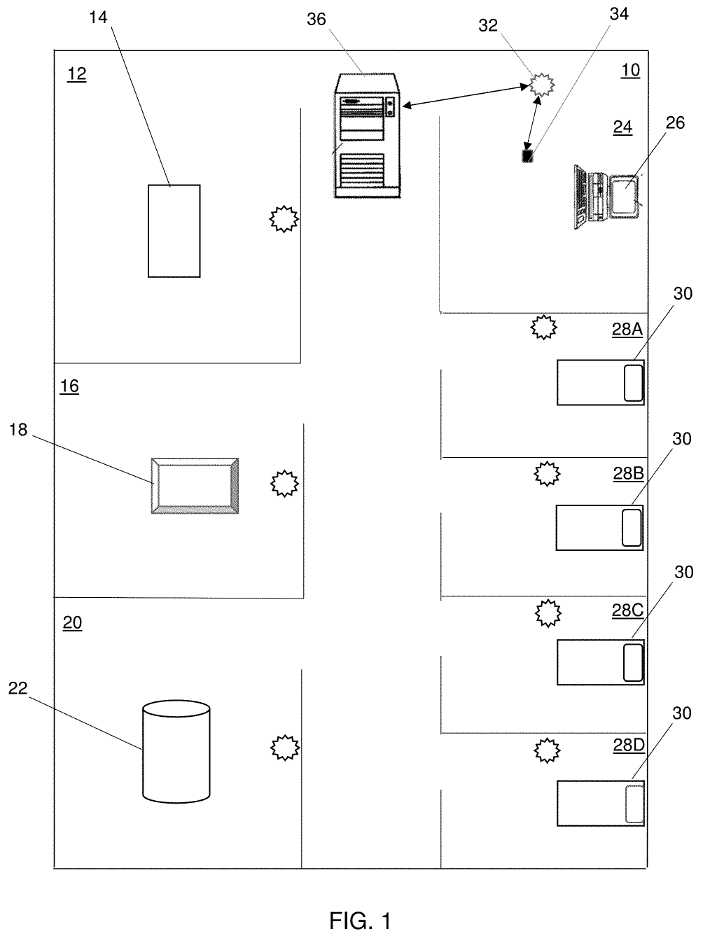

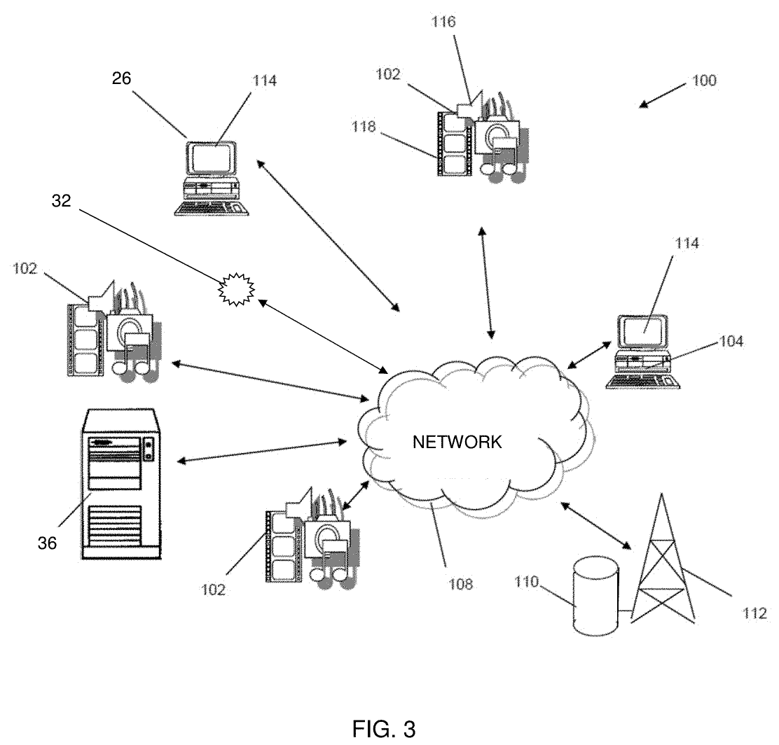

Referring now to the figures, FIG. 1 is a block diagram of a medical facility 10 configured with a system of radio frequency identification (RFID) readers 32 to determine the positions and locations of medical personnel, medical equipment, and patients. The medical facility 10 may have a series of rooms for examining and caring for patients. In the simplified floor plan of the medical facility 10 the following rooms are shown: an operating room 12 with an operating table 14, an x-ray room 16 with an x-ray machine 18, an magnetic resonance imaging (Mill) room 20 with an Mill machine 22, a radiology and imaging room 24 to read/review x-ray and Mill results on a computer and display unit 26, and a series of patient examining rooms 28A-28D, Each of the rooms (12, 16, 20, 24, 28A-28D) have a RFID sensor 32 that is able to read RFID tags 34 assigned or affixed to healthcare workers, patients, and equipment. The RFID sensors communicate via wired or wireless with a computer controller 36 which may have internal storage or is networked with remote storage as will be explained further in FIG. 3.

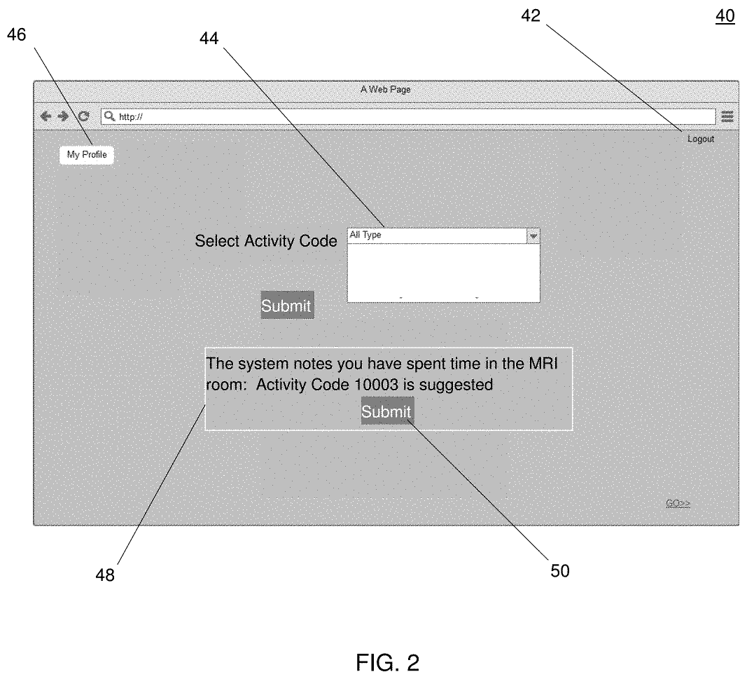

FIG. 2 illustrates an inventive embodiment of a user interface (UI) page 40 for entering medical tasks and corresponding billing codes. A user may login via link 42 or adjust their personal profile with link 46. Based on the location of the healthcare user, an informed and intelligent suggestion may be provided to the healthcare user in field 48. If the suggestion is correct the healthcare user clicks on or selects the submit button 50. In addition, a pull down menu 44 of activities and tasks and corresponding billing codes are also provided with the UI page 40. It is appreciated that the layout and format of the UI page may differ depending on the display device that may illustratively include a smartphone screen, tablet screen, laptop screen, or a computer monitor screen.

FIG. 3 is a schematic diagram illustrating an overall view of communication devices, computing devices, and mediums for implementing a system and method that improves the timeliness, convenience, and accuracy of capturing information for medical billing.

The system 100 includes multimedia devices 102 and desktop computer devices 104 configured with display capabilities 114 and processors for executing instructions and commands, as well as running software. The multimedia devices 102 are optionally mobile communication and entertainment devices, such as cellular phones, tablets, laptops, and mobile computing devices that in certain embodiments are wirelessly connected to a network 108. The multimedia devices 102 typically have video displays 118 and audio outputs 116. RFID sensors 36 may also be in communication with the network 108. RFID sensors 32 may transmit a signal to passive RFID tags or receive signals from active RFID tags. The multimedia devices 102 and desktop computer devices 104 are optionally configured with internal storage, software, and a graphical user interface (GUI) for carrying out elements of the billing information collection system and method according to embodiments of the invention. The network 108 is optionally any type of known network including a fixed wire line network, cable and fiber optics, over the air broadcasts, local area network (LAN), wide area network (WAN), global network (e.g., Internet), intranet, etc. with data/Internet capabilities as represented by server 36. Communication aspects of the network are represented by cellular base station 110 and antenna 112. In a preferred embodiment, the network 108 is a LAN and each remote device 102 and desktop device 104 executes a user interface application (e.g., Web browser) to contact the server system 36 through the network 108. Alternatively, the remote devices 102 and 104 may be implemented using a device programmed primarily for accessing network 108 such as a remote client.

The software for the system and method for medical billing of embodiments of the invention may be resident on tablets 102, desktop or laptop computers 104, or stored within the server 36 or cellular base station 110 for download to an end user. Server 36 may be implemented as a cloud-based service for implementing embodiments of the platform with a multi-tenant database for storage of separate client data for each independent healthcare facility.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. An automated system for medical billing, said system comprising:
one or more radio frequency identification (RFID) sensors positioned in various rooms or areas of a medical facility in communication with:
a first set of individually coded radio frequency identification (RFID) tags assigned to each member of a team of healthcare workers in the medical facility,
a second set of individually coded RFID tags assigned to one or more pieces of equipment in the medical facility,
a third set of individually coded RFID tags assigned to one or more patients seeking medical care in the medical facility; and
a software platform run on a processor that collects information from the one or more RFID sensors to determine probable tasks performed by the members of the healthcare team based on the locations and time spent by the healthcare members with the one or more pieces of equipment and the one or more patients being attended to, and where the determined probable tasks are associated with a corresponding billing codes.

2. The system of claim 1 further comprising a user interface (UI) generated by the software platform that provides informed billing suggestions to healthcare workers.

3. The system of claim 3 wherein the UI is formatted for display on a smartphone, a tablet, a laptop, and a monitor connected to a computer.

4. The system of claim 1 wherein the software platform further comprises artificial intelligence (AI) tools to provide intelligent suggestions as to the probable tasks with corresponding billing codes as performed by members of the healthcare team.

5. A method for medical billing comprising:
a healthcare worker from the team of healthcare workers logging on to the software platform of claim 1; and
reviewing a received informed billing suggestion on a user interface (UI) generated by the software platform and accepting, editing. or declining the informed billing suggestion.

6. The method of claim 5 wherein if the informed billing suggestion is declined, the healthcare worker uses a pull-down menu to select a task and a corresponding billing code.
